(19) United States
(12) Patent Application Publication
(10) Pub. No.: US 2023/0098887 A1
(43) Pub. Date: Mar. 30, 2023

(54) MEDICAL BAG SYSTEM AND CENTRIFUGAL SEPARATION SYSTEM

(71) Applicant: TERUMO KABUSHIKI KAISHA, Tokyo (JP)

(72) Inventor: Yuusuke Yamazaki, Tokyo (JP)

(21) Appl. No.: 18/077,078

(22) Filed: Dec. 7, 2022

Related U.S. Application Data
(63) Continuation of application No. PCT/JP2021/024274, filed on Jun. 28, 2021.

(30) Foreign Application Priority Data
Jun. 30, 2020 (JP) 2020-112747

(51) Int. Cl. A61M 1/02

(52) U.S. Cl. CPC A61M 1/0272; A61M 1/0209; A61J 1/10

(57) ABSTRACT

A medical bag system and a centrifugal separation system are equipped with a main bag in which blood is accommodated, a blood plasma bag in which blood plasma is accommodated, a chemical solution bag in which a red blood cell preservative solution is accommodated, and a transfer tube connecting the main bag, the blood plasma bag, and the chemical solution bag. The chemical solution bag includes a port side end portion having a connection port to which the transfer tube is connected, a suspended side end portion, which is an end portion opposite to the port side end portion, and which is formed with a suspension hole through which a suspension hook is inserted, and further including extension members that project out in a tongue shape from side portions of the suspension hole.

### CROSS-REFERENCE TO RELATED APPLICATIONS

The present disclosure is a continuation of and claims benefit to PCT/JP2021/024274 filed on Jun. 28, 2021, entitled “MEDICAL BAG SYSTEM AND CENTRIFUGAL SEPARATION SYSTEM” which claims priority to Japanese Patent Application No. 2020-112747A filed on Jun. 30, 2020. The entire disclosures of the applications listed above are hereby incorporated herein by reference, in their entireties, for all that they teach and for all purposes.

### BACKGROUND

The present disclosure relates to a medical bag system and a centrifugal separation system which are used in performing centrifugal separation of blood.

In carrying out centrifugal separation of blood, a medical bag system is used having a main bag in which blood is accommodated, a blood plasma bag in which blood plasma is accommodated, and a chemical solution bag in which a red blood cell preservative solution is accommodated (see, for example, Japanese Patent Application No. JPH09-168585 A). The medical bag system is set in a centrifugal separation device, and by applying a centrifugal force to the main bag, whole blood in the main bag is separated into packed (e.g., concentrated) red blood cells and blood plasma. Then, the centrifugal separation device transfers the blood plasma into a blood plasma bag via a tube that is connected to the main bag. Thereafter, the medical bag system is taken out from the centrifugal separation device, and the red blood cell preservative solution in the chemical solution bag is transferred into the main bag in which the packed red blood cells are stored.

### BRIEF SUMMARY

In the centrifugal separation device, a plurality of pockets in which various types of bags are accommodated, and grooves in which tubes that connect the plurality of bags are accommodated are provided on a centrifugal drum which is a rotating portion. The medical bag system is set in the centrifugal separation device in a state with the bags being accommodated in the pockets.

Within such a medical bag system, the bags are arranged with ports that are connected to the tubes facing toward a downward side. However, the pockets of the centrifugal drum are formed in a slit shape which is narrow in the thickness direction of the bags, which makes it difficult to insert one's fingers between the pockets and the bags. Therefore, a problem arises in that it is difficult for an operator to take the bags out of the pockets of the centrifugal drum after completion of the centrifugal separation process.

Further, since the bags contain a chemical solution or blood components, a situation may occur in which the bags sink down into the bottom inner sides of the pockets due to their own weight. In such a case, it is difficult to pick up the bags by hand from the bottom inner sides of the narrow slit-shaped pockets, and the efficiency in performing such an operation is lowered.

On the other hand, in the vicinity of the pockets, a groove for accommodating a tube of the medical bag system is arranged so as to bypass the pockets. The tube, which is arranged in such a groove, may rise up during the occurrence of centrifugal separation, and may come into contact with a fixed portion such as a lid or the like, and may become damaged due to scratching or abrasion, and therefore, a structure for protecting the tube that is in close proximity to the bags is required.

Thus, the present disclosure has at least one object of providing a medical bag system and a centrifugal separation system, in which taking the bags from out of the pockets can be easily performed, together with enabling the tube to be protected in the vicinity of the bags.

One aspect of the present disclosure is characterized by a medical bag system including a bag, and a tube connected to the bag, the medical bag being configured to be used by being set in a centrifugal separation device, wherein the bag includes a connection side end portion to which the tube is connected, a suspended side end portion, which is an end opposite to the connection side end portion, and in which there is formed a suspension hole through which a suspension hook is inserted, and an extension member that projects out in a tongue shape from at least one side in a widthwise direction of the bag, of the suspended side end portion, with respect to a central portion of the suspended side end portion in which the suspension hole is formed.

Another aspect of the present disclosure is characterized by a centrifugal separation system, including the medical bag system according to the above-described aspect, and a centrifugal drum on which the medical bag system is mounted, wherein the centrifugal drum includes a pocket configured to accommodate the bag, and a tube guiding groove configured to accommodate the tube in a portion adjacent to the bag in a direction of centrifugation, wherein the extension member of the bag covers the tube of the tube guiding groove, and a distal end of the extension member is sandwiched and retained in a gap between the tube and the tube guiding groove.

The medical bag system and the centrifugal separation system of the aforementioned aspects make it easy to take the bags from out of the pockets, together with protecting the tube in the vicinity of the bags.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages are described herein and will be apparent to those skilled in the art upon consideration of the following Detailed Description and in view of the figures.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a schematic plan view of a medical bag system according to a first embodiment of the present disclosure;

FIG. 2 is a plan view of a chemical solution bag of the medical bag system shown in FIG. 1;

FIG. 3 is an explanatory diagram showing a configuration of a centrifugal separation device and the medical bag system shown in FIG. 1;

FIG. 4 is a perspective view showing a unit setting area of the centrifugal separation device shown in FIG. 3;

FIG. 5 is a top plan view, showing a state in which the medical bag system is attached to the unit setting area of the centrifugal separation device in accordance with embodiments of the present disclosure;

FIG. 6 is a first perspective view showing an operation of an extension member of the chemical solution bag shown in FIG. 3;

FIG. 7 is a second perspective view showing an operation of the extension member of the chemical solution bag shown in FIG. 3;

FIG. 8 is a perspective view showing a state in which a chemical solution bag according to a comparative example is attached to the centrifugal separation device in accordance with embodiments of the present disclosure;

FIG. 9A is an explanatory diagram of a chemical solution bag of a medical bag system according to a second embodiment of the present disclosure;

FIG. 9B is a side view of the chemical solution bag of FIG. 9A; and

FIG. 10 is a perspective view showing operations of the chemical solution bag of the medical bag system according to the second embodiment of the present disclosure.

### DETAILED DESCRIPTION

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The ensuing description provides embodiments only, and is not intended to limit the scope, applicability, or configuration of the claims. Rather, the ensuing description will provide those skilled in the art with an enabling description for implementing the described embodiments. It being understood that various changes may be made in the function and arrangement of elements without departing from the spirit and scope of the appended claims.

Various aspects of the present disclosure will be described herein with reference to drawings that may be schematic illustrations of idealized configurations.

Hereinafter, embodiments of a medical bag system and a centrifugal separation system will be presented and described in detail below with reference to the accompanying drawings.

### First Embodiment

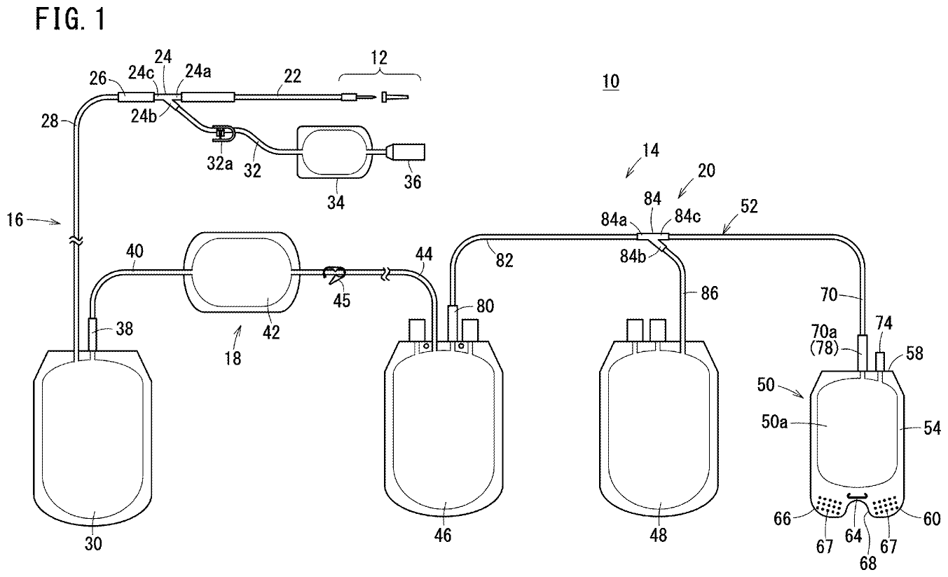

A medical bag system 10, as shown in FIG. 1, is used for collecting blood from a donor. The medical bag system 10 may be used to centrifugally separate blood (e.g., whole blood) collected from the donor into blood components having different specific gravities, namely, platelet poor plasma (PPP) and packed (e.g., concentrated) red blood cells (RBC), and to store the respective blood components in different bags (e.g., of the medical bag system 10).

The medical bag system 10 is equipped with a blood collection needle 12, and a blood bag set 14 to which the blood collection needle 12 is connected. The blood bag set 14 comprises a blood collection unit 16 or portion, a preprocessing unit 18 or portion, and a separation processing unit 20 or portion. The blood collection unit 16 includes a first blood collection tube 22, a branch connector 24, a sealing member 26, a second blood collection tube 28, a blood collection bag 30, a branching tube 32, and an initial flow blood bag 34.

The blood collection needle 12 is connected to one end of the first blood collection tube 22. The other end of the first blood collection tube 22 is connected to the branch connector 24. The branch connector 24 branches in three directions. A first port 24a thereof is connected to the first blood collection tube 22, a second port 24b thereof is connected to the initial flow blood bag 34 via the branching tube 32, and a third port 24c thereof is connected to the sealing member 26. The sealing member 26 is capable of being broken (e.g., opened, unsealed, etc.), and when a breaking operation is performed thereon, the first blood collection tube 22 and the second blood collection tube 28 are placed in communication with each other.

The initial flow blood bag 34 is a bag for collecting a predetermined amount of the initially flowing blood collected from the donor, and a sampling port 36, to which a blood collection tube is capable of being attached and detached, is disposed at one end thereof. A clamp 32a is provided on the branching tube 32, and an opening/closing operation of the branching tube 32 can be carried out through the clamp 32a.

The blood collection bag 30 is connected to another end of the second blood collection tube 28. The blood collection bag 30 is a bag in which whole blood collected from the donor is accommodated. A blood preservative solution such as an anticoagulant or the like is accommodated beforehand inside the blood collection bag 30. The preprocessing unit 18 is connected to the blood collection bag 30. The blood collection bag 30 and the initial flow blood bag 34 are formed in a bag shape by superimposing or overlapping thermoplastic resin sheets that possess flexibility, and fusion bonding peripheral edge portions thereof (e.g., thereby forming a fluid or blood receiving volume that is disposed between the thermoplastic resin sheets within a periphery of the fusion bonded peripheral edge portions, etc.).

The preprocessing unit 18 includes a sealing member 38, an inlet side tube 40, a filter 42, and an outlet side tube 44. The blood collection bag 30 and the inlet side tube 40 are connected via the sealing member 38. The sealing member 38 is a member that is capable of being broken (e.g., opened, unsealed, etc.), and until a breaking operation thereon is performed, serves to prevent the blood collected in the blood collection bag 30 from being transferred into the preprocessing unit 18. Another end of the inlet side tube 40 is connected to the filter 42.

The filter 42, for example, may correspond to a leukocyte removal filter that removes, or is configured to remove, leukocytes contained in the blood. The separation processing unit 20 is connected to an outlet side of the filter 42.

The separation processing unit 20 includes the outlet side tube 44, a main bag 46, a blood plasma bag 48, a chemical solution bag 50, and a transfer tube 52. The main bag 46, the blood plasma bag 48, and the chemical solution bag 50, in the same manner as the blood collection bag 30, are formed by superimposing two resin sheets, and fusion bonding peripheral edge portions thereof (e.g., thereby forming a fluid receiving volume that is disposed between the two resin sheets within a periphery of the fusion bonded peripheral edge portions, etc.).

The outlet side tube 44 is a conduit that connects the main bag 46 and an outlet side of the filter 42, and guides the blood components, from which the leukocytes have been removed by the filter 42, into the main bag 46. A clamp 45 is provided on the outlet side tube 44. By operation of the clamp 45, the user is capable of carrying out an opening/closing operation on the outlet side tube 44.

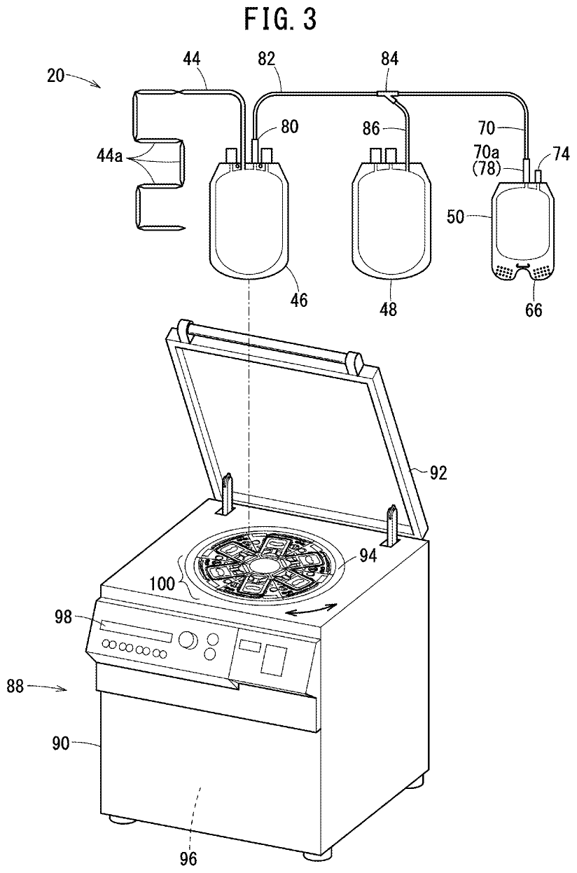

As shown in FIG. 3, after transferring of the blood components into the main bag 46 is completed, the outlet side tube 44 is aseptically separated (e.g., heat sealed and then cut, etc.) from the filter 42. Furthermore, by being sealed at each of intervals of a constant length, the outlet side tube 44 may be formed into a series of tube segments 44a. The tube segments 44a are cut off by the user as needed, and are used for blood testing.

As shown in FIG. 1, the main bag 46 accommodates (e.g., stores, holds, etc.) blood components remaining after the leukocytes have been removed from the blood collected from the donor, by the filter 42. Further, the main bag 46 serves in a dual manner as a red blood cell bag for storing packed red blood cells (RBC) which are obtained after having subjected the blood components to centrifugal separation.

The blood plasma bag 48 is a bag for accommodating and storing blood plasma (e.g., platelet poor plasma, or PPP) which is obtained by subjecting the blood components in the main bag 46 to centrifugal separation. The chemical solution bag 50 is a bag in which a red blood cell preservative solution (for example, a mannitol-adenine-phosphate, or MAP, solution) is accommodated.

The main bag 46, the blood plasma bag 48, and the chemical solution bag 50 may be formed in a bag shape by, for example, superimposing resin sheets, and sealing peripheral edge portions thereof, as described herein. Labels on which predetermined information is printed are affixed to outer surfaces of the main bag 46, the blood plasma bag 48, and the chemical solution bag 50.

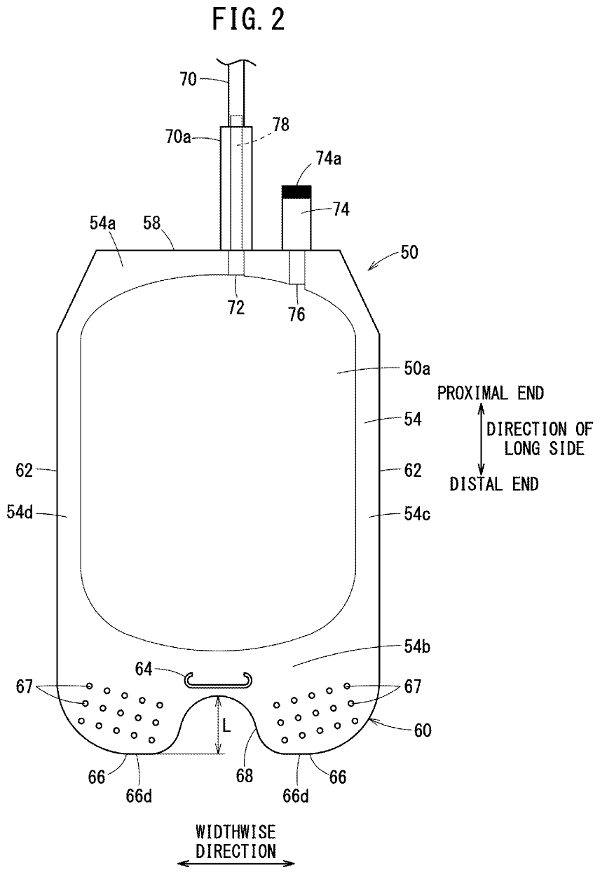

As shown in FIG. 2, as for the chemical solution bag 50, a sealed part 54 is formed thereon, which is formed by fusion bonding the peripheral edge portions of two resin sheets. A storage space 50a (e.g., receiving volume, etc.) of the chemical solution bag 50 is formed in a portion that is surrounded by the sealed part 54, and a chemical solution such as a red blood cell preservative solution or the like is stored in the interior of the storage space 50a. The sealed part 54 includes a first sealed part 54a constituting a port side end portion 58 in which there is formed a connection port 72 for connection with a tube, a second sealed part 54b constituting a suspended side end portion 60 of the chemical solution bag 50, and a third sealed part 54c and a fourth sealed part 54d that make up sides 62 of the chemical solution bag 50. On the suspended side end portion 60 in which the second sealed part 54b is formed, a suspension hole 64 (e.g., aperture, slot, etc.) may be formed in a center of the chemical solution bag 50 in a widthwise direction. The suspension hole 64 may be used to suspend the chemical solution bag 50. The suspension hole 64 is shown in the form, or shape, of a C-shaped cutout, that is extended in a narrow elongated manner in the widthwise direction.

Further, a pair of extension members 66, or tabs, which project out in a tongue shape toward an opposite side (e.g., a distal end side in a direction of the long side) from the port side end portion 58, are formed on both side portions of the suspension hole 64 of the suspended side end portion 60. The extension members 66 are formed by increasing the dimension of the second sealed part 54b in the direction of the long side. A recessed part 68 is formed between the extension members 66. The projecting length L at which the extension members 66 project out with respect to the recessed part 68 is formed in a length that makes it easy for the user to pinch the extension members 66 with the fingers. The projecting length L is preferably formed to be longer than one half the circumference of an outer circumference of the tube that constitutes the transfer tube 52. Further, on surfaces of the extension members 66, a concave and convex pattern 67 is formed that functions as a non-slip surface when pinched by the user with the fingers. In some embodiments, the concave and convex pattern 67 may correspond to dimples formed in one or more of the resin sheets that form the chemical solution bag 50. Theses dimples may include a domed portion of the resin sheets that extend in a direction into and/or out of the page, as shown in FIG. 2. For instance, the dimples may include a plurality of domed convex portions extending from a first resin sheet in a direction away from a second resin sheet of the chemical solution bag 50, or vice versa. Other shapes of the concave and convex pattern 67 may be used without limiting the scope of the present disclosure (e.g., one or more embossed and/or debossed ridges, linear shapes, arcuate shapes, etc., and/or combinations thereof).

On the port side end portion 58 of the chemical solution bag 50, there are provided the connection port 72 to which a later-described third tube 70 (see, e.g., FIG. 1) of the transfer tube 52 is connected, and a filling port 76 to which a filling tube 74 is connected. The filling tube 74 is used when storing a chemical solution in the storage space 50a, and is cut and sealed after the chemical solution has been filled. In a state in which the medical bag system 10 is provided as a product, the filling tube 74 is cut short and is sealed by a sealing member 74a.

When the sealed part 54 is formed, the connection port 72 and the filling port 76 are fusion bonded in a state of being placed in communication with the storage space 50a. The third tube 70 which is connected to the connection port 72 is equipped with a cylindrical end structure 70a for connection to the connection port 72 at an end thereof, and a sealing member 78 is provided in the interior of the end structure 70a.

As shown in FIG. 1, the transfer tube 52 connects the main bag 46, the blood plasma bag 48, and the chemical solution bag 50. The transfer tube 52 includes a sealing member 80, a first tube 82, a branch connector 84, a second tube 86, the third tube 70, and a sealing member 78. The sealing member 80 connects the main bag 46 and one end of the first tube 82 to each other. The sealing member 80 is a member that is capable of being broken (e.g., opened, unsealed, etc.) in a similar manner to the sealing member 26, and until a breaking operation is performed thereon, prevents communication between the main bag 46 and the first tube 82.

The other end of the first tube 82 is connected to a first port 84a of the branch connector 84. The branch connector 84 is a connector which is branched in three directions defined by a first port 84a, a second port 84b, and a third port 84c. One end of the second tube 86 is connected to the second port 84b of the branch connector 84, and the third tube 70 is connected to the third port 84c thereof.

The other end of the second tube 86 is connected to the blood plasma bag 48. The other end of the third tube 70 is connected to the sealing member 78. The sealing member 78 is provided at the port side end portion 58 of the chemical solution bag 50, and connects the chemical solution bag 50 and the third tube 70. The sealing member 78 blocks communication between the chemical solution bag 50 and the third tube 70 until the breaking operation is performed, and prevents the red blood cell preservative solution of the chemical solution bag 50 from flowing into the third tube 70.

As shown in FIG. 3, in the medical bag system 10, after collection of blood and preprocessing by the filter 42 are completed, the outlet side tube 44 is cut. Then, the separation processing unit 20 of the medical bag system 10 is cut and separated away from the blood collection unit 16 and the preprocessing unit 18. Thereafter, the separation processing unit 20 is set in a centrifugal separation device 88, and a centrifugal separation process is carried out.

The centrifugal separation device 88 includes a box-shaped casing 90, a lid member 92 that is capable of opening and closing an upper part of the casing 90, and a centrifugal drum 94 provided in the casing 90. In the casing 90 of the centrifugal separation device 88, there are provided a motor (not shown) for causing the centrifugal drum 94 to rotate, a control unit 96 (e.g., controller or processor, etc.) for controlling operations of the centrifugal separation device 88, and a display input unit 98 (e.g., display device, touchscreen display, etc.) that receives display of data to the user and operations that are input from the user.

The centrifugal drum 94 includes six unit setting areas 100 in which the separation processing unit 20 can be set. One of the unit setting areas 100 is set within a range occupying 600 with respect to the center of rotation of the centrifugal drum 94, and the unit setting areas 100 are arranged alongside one another without gaps along the circumferential direction. In some embodiments, each of the unit setting areas 100 may be arranged angularly equidistant from one another (e.g., at equal angular intervals, etc.) around a center (e.g., center rotational axis, etc.) of the centrifugal drum 94.

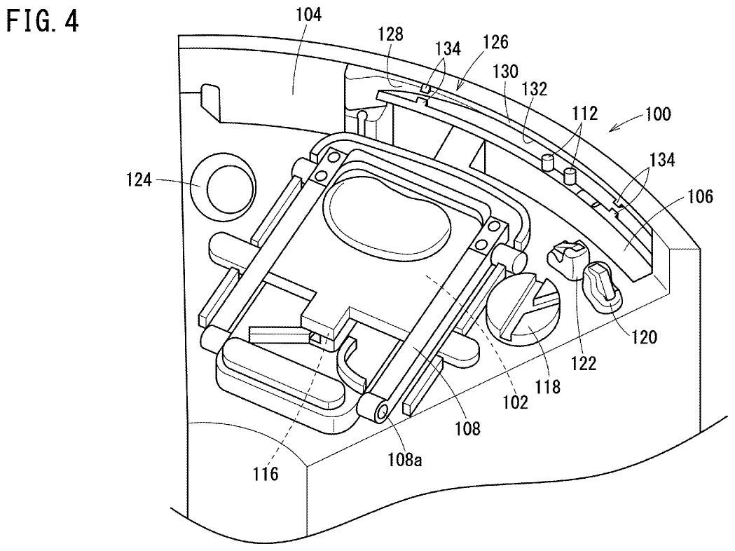

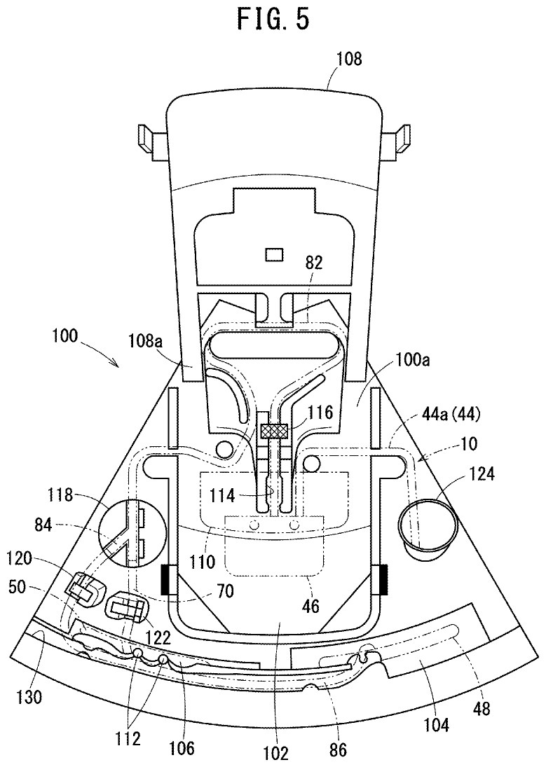

As shown in FIGS. 4 and 5, a main bag pocket 102 in which the main bag 46 is accommodated, a blood plasma bag pocket 104 in which the blood plasma bag 48 is accommodated, and a chemical solution bag pocket 106 in which the chemical solution bag 50 is accommodated are formed in each of the unit setting areas 100. As shown in FIG. 5, the main bag pocket 102 is disposed in closer proximity to a center portion in a radial direction of the unit setting area 100. A lid member 108 that is capable of being opened and closed is provided on the main bag pocket 102, and as shown in FIG. 4, the centrifugal separation process is carried out in a state with the lid member 108 being closed. As shown in FIG. 5, the main bag pocket 102 has a larger volume than the blood plasma bag pocket 104 and the chemical solution bag pocket 106.

A pressing element 110 for compressing the main bag 46 and pushing out the separated blood components is provided on an inner peripheral side portion of the main bag pocket 102. A surface of the pressing element 110 that faces toward the main bag 46 is formed by an inclined surface. The pressing element 110 advances and retracts along a radial direction of the centrifugal drum 94 under the control of the control unit 96. In some embodiments, the pressing element 110 may correspond to a linear actuator, solenoid, motor-and-cam, and/or some other mechanism that is configured to selectively move into engagement with, and apply a mechanical force to, a portion of the main bag 46 that is disposed in the main bag pocket 102.

As shown in FIG. 5, the blood plasma bag pocket 104 and the chemical solution bag pocket 106 are arranged alongside one another in the circumferential direction, on an outer peripheral side in the radial direction of the main bag pocket 102. The chemical solution bag pocket 106 is formed in a narrow elongated groove shape having a circumferential dimension longer than a radial dimension thereof. The volume of the chemical solution bag pocket 106 is formed to be smaller than the volume of the blood plasma bag pocket 104, and a dimension in the radial direction of the chemical solution bag pocket 106 is smaller than the dimension in the radial direction of the blood plasma bag pocket 104. A suspension hook 112 in order for the suspension hole 64 of the chemical solution bag 50 to be suspended is provided on an edge on the outer peripheral side of the chemical solution bag pocket 106. Stated another way, the suspension hook 112 may be at least partially inserted into, or through, the suspension hole 64 of the chemical solution bag 50 such that the chemical solution bag 50 is held suspended in the chemical solution bag pocket 106.

The first tube 82, the second tube 86, the third tube 70, and the tube segments 44a that constitute the outlet side tube 44 and the transfer tube 52 of the medical bag system 10 are arranged on an upper surface 100a of the unit setting area 100. On the upper surface 100a, the first tube 82 and one portion of the tube segments 44a are arranged radially on a more inward side in the radial direction than the main bag pocket 102.

Further, a hinge part 108a of the lid member 108 that covers the main bag pocket 102 is provided on the upper surface 100a. The lid member 108 is arranged so as to cover a part of the first tube 82. A breaking member 114 that serves to break the sealing member 80, and an infrared sensor 116 which detects the blood components flowing through the first tube 82 are disposed at a portion where the first tube 82 that is covered by the lid member 108 is arranged.

Further, on one side portion of the upper surface 100a, a holder 118 in which the first tube 82, the branch connector 84, the second tube 86, and the third tube 70 are retained, a blood plasma clamp 120 for opening and closing the second tube 86, and a chemical solution clamp 122 for opening and closing the third tube 70 are provided. On the other side portion of the upper surface 100a, a segment pocket 124 is provided in which a non-illustrated segment holder is accommodated in which the tube segments 44a are bundled.

As shown in FIG. 4, a tube retaining section 126, which projects upwardly from the upper surface 100a, is provided on the outer peripheral side of the chemical solution bag pocket 106. The tube retaining section 126 includes an outer wall 128 connected to an outer circumferential edge of the unit setting area 100, and an inner wall 130 that projects out from the outer circumferential edge further toward an inward side than the outer wall 128. A tube guiding groove 132 in which the second tube 86 is accommodated is formed between the outer wall 128 and the inner wall 130. The inner wall 130 extends from the vicinity of the blood plasma clamp 120, and through the outer circumference of the chemical solution bag pocket 106 until reaching the vicinity of a midway location in the circumferential direction of the blood plasma bag pocket 104, and serves to guide the second tube 86 to the blood plasma bag pocket 104. Further, in order to prevent the second tube 86 from falling out from the tube guiding groove 132, guide pieces 134 are disposed at predetermined sites of the tube guiding groove 132. The guide pieces 134 may correspond to retention fingers arranged at an opening of the tube guiding groove 132. These retention fingers may be disposed on opposite sides of the tube guiding groove 132 extending toward one another and closing a portion of the gap of the tube guiding groove 132. In some embodiments, the second tube 86 may need to be compressed at each of the guide pieces 134 to be inserted into the tube guiding groove 132. Once inserted into the tube guiding groove 132, the second tube 86 may be retained in the tube guiding groove 132 by the retention fingers until the second tube 86 is manually compressed at each of the guide pieces 134 and the second tube 86 is removed from the tube guiding groove 132.

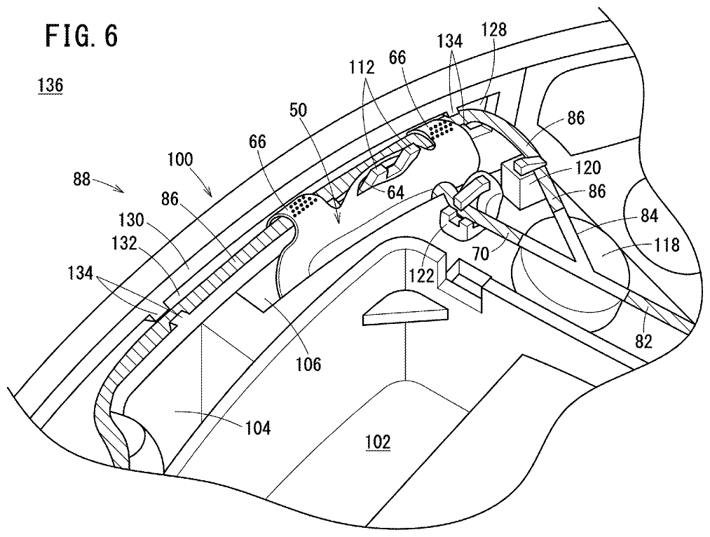

As shown in FIG. 6, a centrifugal separation system 136 according to the present embodiment comprises the medical bag system 10 and a centrifugal separation device 88 in which the medical bag system 10 has been set.

The medical bag system 10 and the centrifugal separation system 136 according to the present embodiment are configured in the manner described above, and next, operations thereof will be described below.

In the medical bag system 10 shown in FIG. 1, blood which is collected from the donor by a medical worker at a blood collection site is stored in the blood collection bag 30. Thereafter, the medical bag system 10 is transported to a blood center. At the blood center, the preprocessing unit 18 is utilized by the user in order to remove leukocytes from whole blood, and to store in the main bag 46 blood components from which the leukocytes have been removed.

Thereafter, in order for the user to subject the blood components of the main bag 46 to centrifugal separation, the outlet side tube 44 is severed, and the separation processing unit 20 is cut and separated away from the preprocessing unit 18. In addition, the separation processing unit 20 is set in the centrifugal separation device 88.

As shown in FIG. 5, in one of the unit setting areas 100 of the centrifugal separation device 88, the main bag 46 is set in the main bag pocket 102, an empty blood plasma bag 48 is set in the blood plasma bag pocket 104, and the chemical solution bag 50 is set in the chemical solution bag pocket 106 by the user. Further, the first tube 82, the second tube 86, and the third tube 70 are arranged along the upper surface 100a of the unit setting area 100.

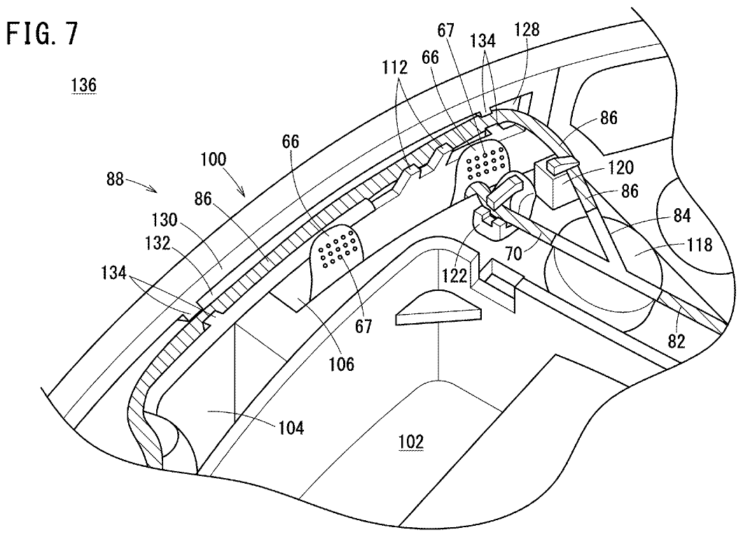

As shown in FIG. 6, the chemical solution bag 50 is inserted into the chemical solution bag pocket 106, in a state with the suspended side end portion 60 on which the extension members 66 are formed facing upward, and with the port side end portion 58 facing downward. In addition, the user passes the suspension hook 112 through the suspension hole 64 of the chemical solution bag 50, thereby fixing the chemical solution bag 50 in the chemical solution bag pocket 106. When the chemical solution bag 50 is set in the chemical solution bag pocket 106, a situation is considered in which the user may mistakenly drop the chemical solution bag 50 into the chemical solution bag pocket 106, in the manner shown in FIG. 7. Since the chemical solution bag pocket 106 has a narrow dimension in the radial direction, if the chemical solution bag 50 disadvantageously becomes inserted into the bottom inner side, it may be difficult to grasp and pick up the main body portion of the chemical solution bag 50 with the fingers. Even in such a case, since the extension members 66 extend upwardly on the chemical solution bag 50 according to the present embodiment, one or more of the extension members 66 can be easily grasped or pinched, and the chemical solution bag 50 can easily be pulled out from the chemical solution bag pocket 106. For instance, the overall dimension of the long side of the chemical solution bag 50 (e.g., from the port side end portion 58 to the distal ends 66d of the extension members 66) may be set to be greater than the dimension of the depth of the chemical solution bag pocket 106. In this arrangement, the extension members 66 may extend above the opening of the chemical solution bag pocket 106 (e.g., for user access and retrieval) even if the chemical solution bag 50 drops into the chemical solution bag pocket 106 during loading/unloading.

Next, the user inserts the second tube 86 extending from the blood plasma bag 48 into the tube guiding groove 132. When the second tube 86 is inserted into the tube guiding groove 132, as shown in FIG. 6, the user presses the second tube 86 into the tube guiding groove 132 together with the extension members 66, in a state in which the extension members 66 of the chemical solution bag 50 are wrapped around the second tube 86. Consequently, the gap between the second tube 86 and the tube guiding groove 132 is filled and covered by the extension members 66. Due to this, the second tube 86 is securely held in place inside the tube guiding groove 132.

After the user has set the medical bag system 10 in the unit setting area 100 as described above, the centrifugal separation process of the blood components is carried out by the centrifugal separation device 88. The centrifugal separation device 88 causes the centrifugal drum 94 to rotate under the control of the control unit 96, and thereby centrifugally separates the blood components in the main bag 46 into platelet poor plasma and packed red blood cells having different specific gravities. Thereafter, the centrifugal separation device 88 applies pressure to the main bag 46 with the pressing element 110. Consequently, the platelet poor plasma with a lower specific gravity flows out from the main bag 46, flows sequentially through the first tube 82, the branch connector 84, and the second tube 86, and is transferred into the blood plasma bag 48.

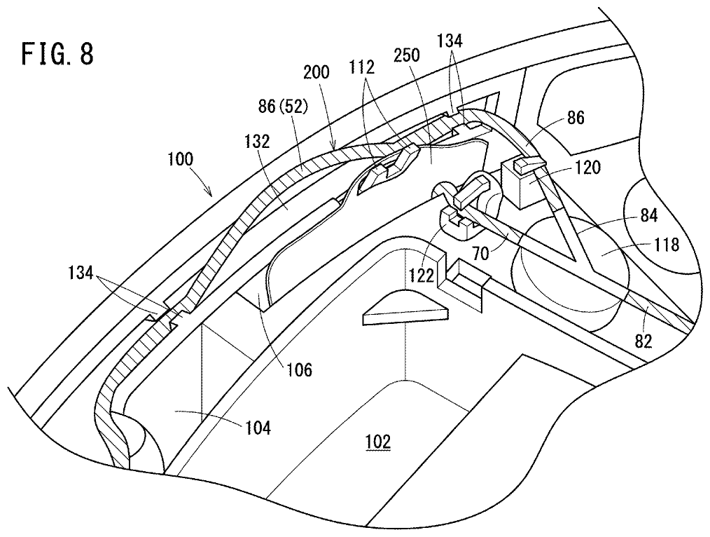

With the medical bag system 200 according to a comparative example shown in FIG. 8, while centrifugal separation is being carried out by the centrifugal separation device 88, the second tube 86 rises up from the tube guiding groove 132, and an event occurs in which the second tube 86 is damaged by scraping against the lid member 92 (see, e.g., FIG. 3) of the centrifugal separation device 88. Due to the occurrence of such an event, the platelet poor plasma which has leaked out from the second tube 86 becomes disadvantageously scattered around the interior of the centrifugal separation device 88.

In contrast thereto, as shown in FIG. 6, according to the medical bag system 10 of the present embodiment, since the gap between the second tube 86 and the tube guiding groove 132 is filled and covered by the extension members 66 of the chemical solution bag 50, it is possible to prevent the second tube 86 from rising up from the tube guiding groove 132. Further, even if the second tube 86 rises up from the tube guiding groove 132, since the outer circumferential portion of the second tube 86 is protected by the extension members 66, instead of the second tube 86, only the extension members 66 are subjected to rubbing or chafing. Consequently, damage due to abrasion or scratching between the second tube 86 and the lid member 92 is prevented.

When the platelet poor plasma is transferred from the main bag 46 into the blood plasma bag 48, the centrifugal separation device 88 retracts the pressing element 110, and causes the rotation of the centrifugal drum 94 to stop, thereby enabling the medical bag system 10 to be taken out. Thereafter, the user takes out the medical bag system 10 from the centrifugal separation device 88. Extraction of the chemical solution bag 50 can be easily performed by pulling out the extension members 66, which are wrapped around the second tube 86 shown in FIG. 6, from the tube guiding groove 132, pinching the extension members 66 with the fingers, and pulling up the chemical solution bag 50 from the chemical solution bag pocket 106.

With the chemical solution bag 250 according to the comparative example shown in FIG. 8, the area over which the chemical solution bag 250 can be pinched with the fingers is narrow, and the operation of taking out the chemical solution bag 250 is made difficult. In contrast thereto, as shown in FIGS. 6 and 7, with the chemical solution bag 50 according to the present embodiment, since the extension members 66 are provided that project out upwardly from the suspended side end portion 60, the operation of taking out the chemical solution bag 50 can be easily performed.

The medical bag system 10 and the centrifugal separation system 136 according to the present embodiment at least provide the following advantageous effects.

The medical bag system 10 of the present embodiment relates to a medical bag system 10 equipped with a bag (e.g., the chemical solution bag 50) and a tube (e.g., the transfer tube 52) that is connected to the bag, and the medical bag system 10 is used by being set in the centrifugal separation device 88. The bag of the medical bag system 10 includes the port side end portion 58 to which the tube is connected, the suspended side end portion 60, which is an end opposite to the port side end portion 58, and in which there is formed the suspension hole 64 through which the suspension hook 112 is inserted, and the extension members 66, which project out in a tongue shape from at least one side in a widthwise direction of the bag, of the suspended side end portion 60, with respect to a central portion of the suspended side end portion 60 in which the suspension hole 64 is formed.

In accordance with the above-described medical bag system 10, since the extension members 66 are provided on both sides of the suspension hole 64, it becomes easy for the operator to perform the operations of mounting the bag (e.g., the chemical solution bag 50) into and taking the bag (e.g., the chemical solution bag 50) from out of the chemical solution bag pocket 106 of the centrifugal separation device 88. Further, since the extension members 66 are capable of covering the outer circumferential portion of the second tube 86 of the transfer tube 52, it is possible to prevent damage from occurring to the second tube 86 (e.g., during centrifugal operation, etc.).

In the above-described medical bag system 10, the extension members 66 may be formed in a manner that the projecting length at which the extension members project out from the suspension hole 64 is longer than one half an outer circumference of the transfer tube 52. In accordance with such a configuration, it becomes easy to attach the extension members 66 so as to wrap around the outer circumference of the second tube 86, and the second tube 86 can be protected more effectively.

In the above-described medical bag system 10, a pair of the extension members 66 may be provided on both sides of the suspension hole 64. In accordance with such a configuration, it becomes easy for the operator to perform the operations of mounting the bag (the chemical solution bag 50) into and taking the bag (e.g., the chemical solution bag 50) from out of the pocket (e.g., the chemical solution bag pocket 106) of the centrifugal separation device 88.

In the above-described medical bag system 10, the non-slip concave and convex pattern 67 may be formed on one or more surfaces of the extension members 66. In accordance with such a configuration, the user can easily pinch the extension members 66, and the operations of mounting and taking out the chemical solution bag 50 into and from the chemical solution bag pocket 106 can be more easily performed.

In the above-described medical bag system 10, the extension members 66 may be formed as fusion bonded portions of the pair of resin sheets that constitute the chemical solution bag 50 and which are superimposed in a thickness direction. In accordance with such a configuration, since the extension members 66 are integrally formed at the same time as the chemical solution bag 50, productivity is superior.

The medical bag system 10 may be the blood bag set 14, which is equipped with the main bag 46 in which blood is accommodated, the blood plasma bag 48 in which blood plasma is accommodated, the chemical solution bag 50 in which the red blood cell preservative solution is accommodated, and the transfer tube 52 connecting the main bag 46, the blood plasma bag 48, and the chemical solution bag 50.

The centrifugal separation system 136 according to the present embodiment is constituted by the centrifugal separation system 136, including the above-described medical bag system 10, and the centrifugal drum 94 on which the medical bag system 10 is mounted. In the centrifugal separation system 136, the centrifugal drum 94 includes the main bag pocket 102 in which the main bag 46 is accommodated, the blood plasma bag pocket 104 in which the blood plasma bag 48 is accommodated, the chemical solution bag pocket 106 in which the chemical solution bag 50 is accommodated, and the tube guiding groove 132 which is provided in a portion adjacent to the chemical solution bag pocket 106 in the direction of centrifugation, and in which the transfer tube 52 is accommodated. In addition, in the chemical solution bag 50 of the medical bag system 10, the extension members 66 cover the second tube 86 of the tube guiding groove 132, and the distal ends 66d of the extension members 66 are sandwiched and retained in the gap between the second tube 86 and the tube guiding groove 132.

The centrifugal separation system 136 having the above-described configuration makes it easy to take out the chemical solution bag 50 from the chemical solution bag pocket 106, together with protecting the transfer tube 52 (the second tube 86) in the vicinity of the chemical solution bag 50, and preventing damage from occurring to the transfer tube 52.

### Second Embodiment

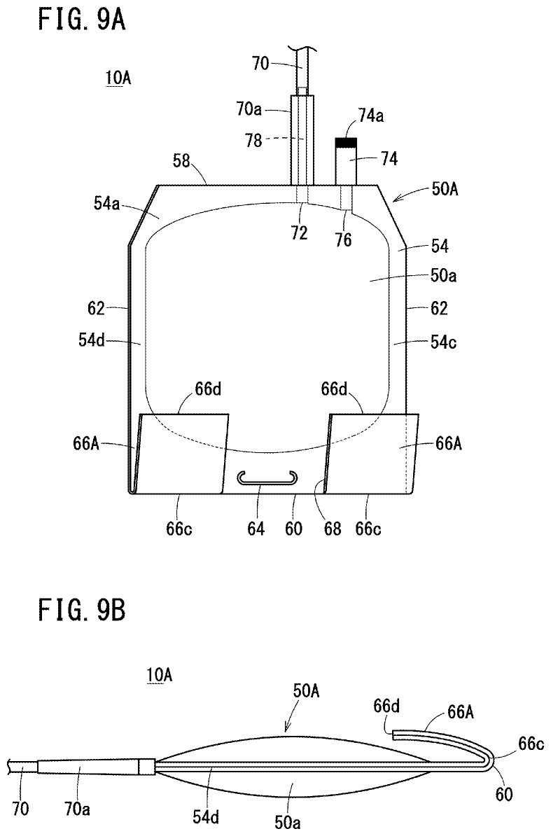

As shown in FIGS. 9A and 9B, the medical bag system 10A of the present embodiment differs from the medical bag system 10 (see, e.g., FIG. 1) of the first embodiment in relation to the chemical solution bag 50A. Structural elements other than those of the chemical solution bag 50A are not shown. Moreover, in the chemical solution bag 50A shown in FIGS. 9A and 9B, structural elements thereof which are the same as those of the chemical solution bag 50 (see, e.g., FIG. 2) of the medical bag system 10 according to the first embodiment are designated by the same reference numerals as with the chemical solution bag 50, and detailed description of such features is omitted.

As shown in FIGS. 9A and 9B, the chemical solution bag 50A is provided with a pair of extension members 66A on the suspended side end portion 60 opposite to the port side end portion 58. The extension members 66A extend out from both side portions of the suspension hole 64. In the same manner as the extension members 66 of the chemical solution bag 50 according to the first embodiment (see, e.g., FIG. 2), the extension members 66A according to the present embodiment are integrally formed with the chemical solution bag 50 by the sealed part 54. However, the extension members 66A of the present embodiment have, at the suspended side end portion 60, folded-back portions 66c which are formed by performing a bending process on the suspended side end portion. Since the extension members 66 are folded back at the folded-back portions 66c, the extension members 66 are arranged in a manner so that the distal ends 66d thereof face toward the port side end portion 58. More specifically, as shown in FIG. 9B, when viewed from the side, the extension members 66A are shaped in an overlapping manner with the storage space 50a of the chemical solution bag 50A.

As described above, with the medical bag system 10A according to the present embodiment, in the chemical solution bag 50A, the extension members 66A are provided with the folded-back portions 66c by which the extension members are folded back toward the port side end portion 58, and the distal ends 66d of the extension members 66A are folded back toward the port side end portion 58.

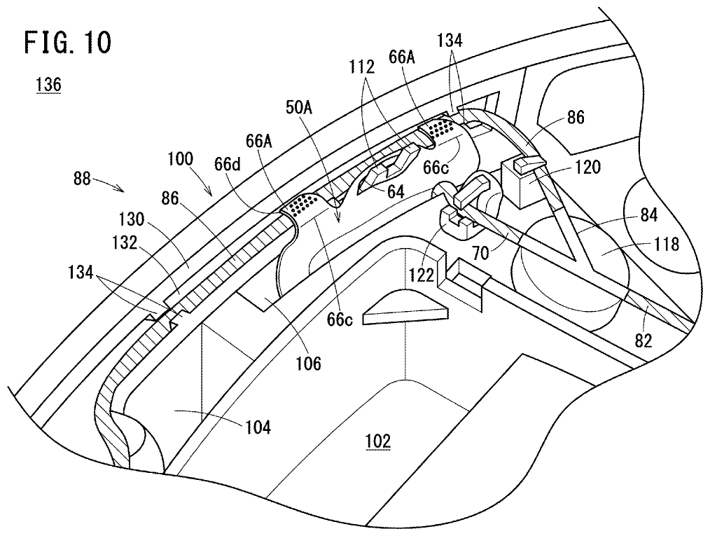

As shown in FIG. 10, according to the above-described chemical solution bag 50A, the extension members 66A are shaped in a manner so as to face toward the port side end portion 58. Therefore, when the chemical solution bag 50A is mounted on the unit setting area 100 of the centrifugal drum 94, it becomes easy to arrange the extension members 66A so as to wrap around the second tube 86 of the transfer tube 52, and the second tube 86 can be protected more effectively.

In some embodiments, the extension members 66 of the medical bag system 10 described in conjunction with the first embodiment may be formed to fold back similarly, if not identical, to the extension members 66A of the medical bag system 10A. For instance, the chemical solution bag 50, may formed in a manner where each of the extension members 66 are folded by a heat forming operation (e.g., by folding the extension members 66 to include a similar bend as shown arranged adjacent the suspended side end portion 60 of the extension members 66A, in FIG. 9B, and then applying heat to the extension members 66 while being held in the folded arrangement, etc.). After the heat forming operation is performed, the extension members 66 may be allowed to cool thereby setting the fold in the extension members 66 of the sealed part 54 of the chemical solution bag 50. The extension members 66A of the medical bag system 10A may be formed in the same, or similar, manner as described above.

Although several embodiments of the present disclosure have been described above, the present disclosure is not limited to the above-described embodiments, and it goes without saying that various modifications can be adopted therein without departing from the scope of the disclosure.

For example, the extension members 66, 66A need not necessarily be integrally formed with the sealed part 54 of the chemical solution bag 50, 50A, and may be formed by joining separate resin sheets by way of fusion bonding to the suspended side end portion 60 of the chemical solution bag 50 or 50A.

Any of the steps, functions, and operations discussed herein can be performed continuously and automatically.

While the flowcharts have been discussed and illustrated in relation to a particular sequence of events, it should be appreciated that changes, additions, and omissions to this sequence can occur without materially affecting the operation of the disclosed embodiments, configuration, and aspects.

The exemplary systems and methods of this disclosure have been described in relation to medical bags and centrifugal separation systems. However, to avoid unnecessarily obscuring the present disclosure, the preceding description omits a number of known structures and devices. This omission is not to be construed as a limitation of the scope of the claimed disclosure. Specific details are set forth to provide an understanding of the present disclosure. It should, however, be appreciated that the present disclosure may be practiced in a variety of ways beyond the specific detail set forth herein.

A number of variations and modifications of the disclosure can be used. It would be possible to provide for some features of the disclosure without providing others.

References in the specification to “one embodiment,” “an embodiment,” “an example embodiment,” “some embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in conjunction with one embodiment, it is submitted that the description of such feature, structure, or characteristic may apply to any other embodiment unless so stated and/or except as will be readily apparent to one skilled in the art from the description. The present disclosure, in various embodiments, configurations, and aspects, includes components, methods, processes, systems and/or apparatus substantially as depicted and described herein, including various embodiments, subcombinations, and subsets thereof. Those of skill in the art will understand how to make and use the systems and methods disclosed herein after understanding the present disclosure. The present disclosure, in various embodiments, configurations, and aspects, includes providing devices and processes in the absence of items not depicted and/or described herein or in various embodiments, configurations, or aspects hereof, including in the absence of such items as may have been used in previous devices or processes, e.g., for improving performance, achieving ease, and/or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments, configurations, or aspects for the purpose of streamlining the disclosure. The features of the embodiments, configurations, or aspects of the disclosure may be combined in alternate embodiments, configurations, or aspects other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment, configuration, or aspect. Thus, the following claims are hereby incorporated into this Detailed Description, with each claim standing on its own as a separate preferred embodiment of the disclosure.

Moreover, though the description of the disclosure has included description of one or more embodiments, configurations, or aspects and certain variations and modifications, other variations, combinations, and modifications are within the scope of the disclosure, e.g., as may be within the skill and knowledge of those in the art, after understanding the present disclosure. It is intended to obtain rights, which include alternative embodiments, configurations, or aspects to the extent permitted, including alternate, interchangeable and/or equivalent structures, functions, ranges, or steps to those claimed, whether or not such alternate, interchangeable and/or equivalent structures, functions, ranges, or steps are disclosed herein, and without intending to publicly dedicate any patentable subject matter.

It is to be appreciated that any feature described herein can be claimed in combination with any other feature(s) as described herein, regardless of whether the features come from the same described embodiment.

As used herein, the singular forms “a,” “an,” and “the” are intended to include the plural forms as well, unless the context clearly indicates otherwise. It will be further understood that the terms “include,” “including,” “includes,” “comprise,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof. The term “and/or” includes any and all combinations of one or more of the associated listed items.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” and “A, B, and/or C” means A alone, B alone, C alone, A and B together, A and C together, B and C together, or A, B, and C together. When each one of A, B, and C in the above expressions refers to an element, such as X, Y, and Z, or a class of elements, such as X₁-Xₙ, Y₁-Yₘ, and Z₁-Zₒ, the phrase is intended to refer to a single element selected from X, Y, and Z, a combination of elements selected from the same class (e.g., X₁ and X₂) as well as a combination of elements selected from two or more classes (e.g., Y₁ and Zₒ).

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and this disclosure.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

What is claimed is:

1. A medical bag system comprising a bag and a tube connected to the bag, the medical bag system being configured to be used by being set in a centrifugal separation device;
wherein the bag comprises:
a port side end portion to which the tube is connected;
a suspended side end portion, which is disposed at an end of the bag opposite to the port side end portion, and in which there is formed a suspension hole; and
an extension member that projects out from the suspended side end portion in a tongue shape from at least one side of the bag in a widthwise direction of the bag with respect to a central portion of the suspended side end portion in which the suspension hole is formed.

2. The medical bag system according to claim 1, wherein the extension member is formed in a manner that a projecting length at which the extension member projects out from the suspension hole is longer than one half an outer circumference of the tube.

3. The medical bag system according to claim 1, wherein the extension member comprises a pair of extension members that extend from each side of the central portion of the suspended side end portion in a direction away from the port side end portion.

4. The medical bag system according to claim 3, wherein a recessed part is formed between the pair of extension members.

5. The medical bag system according to claim 1, wherein a non-slip concave and convex pattern is formed on a surface of the extension member.

6. The medical bag system according to claim 1, wherein the extension member comprises a folded-back portion corresponding to a distal end of the extension member that is folded back toward the port side end portion.

7. The medical bag system according to claim 1, wherein the extension member is formed as a fusion bonded portion of a pair of resin sheets forming the bag and which are superimposed in a thickness direction.

8. The medical bag system according to claim 1, wherein the extension member is formed by joining to the bag a resin sheet that is separate from a resin sheet forming the bag.

9. The medical bag system according to claim 1, further comprising:
a main bag configured to accommodate blood;
a blood plasma bag configured to accommodate blood plasma;
a chemical solution bag configured to accommodate a red blood cell preservative solution; and
a tube configured to connect the main bag, the blood plasma bag, and the chemical solution bag;
wherein the extension member is provided on the chemical solution bag.

10. A centrifugal separation system, comprising:
a medical bag system comprising a bag and a tube connected to the bag, the medical bag system being configured to be used by being set in a centrifugal separation device;
wherein the bag comprises:
a port side end portion to which the tube is connected;
a suspended side end portion, which is disposed at an end of the bag opposite to the port side end portion, and in which there is formed a suspension hole; and
an extension member that projects out from the suspended side end portion in a tongue shape from at least one side of the bag in a widthwise direction of the bag with respect to a central portion of the suspended side end portion in which the suspension hole is formed; and
a centrifugal drum on which the medical bag system is mounted;
wherein the centrifugal drum includes:
a pocket configured to accommodate the bag; and
a tube guiding groove configured to accommodate the tube in a portion adjacent to the bag in a direction of centrifugation;
wherein the extension member of the bag covers the tube of the tube guiding groove, and a distal end of the extension member is sandwiched and retained in a gap between the tube and the tube guiding groove.

11. A medical bag, comprising:
a first resin sheet defining an area of the medical bag extending a length from a proximal end of the medical bag to a distal end of the medical bag and extending a width from a first side of the medical bag to a second side of the medical bag;
a second resin sheet arranged overlapping the first resin sheet, wherein the first resin sheet is bonded to the second resin sheet along a bonded edge that follows at least a portion of a periphery of the area of the medical bag, and wherein a fluid receiving volume is disposed between the first resin sheet and the second resin sheet inside the area of the medical bag;
a port side end portion disposed at the proximal end of the medical bag;
at least one tube extending from the proximal end of the medical bag in a direction away from the distal end of the medical bag, wherein the at least one tube is in fluid communication with the fluid receiving volume;
a suspended side end portion disposed at the distal end of the medical bag, the suspended side end portion comprising a sealed area of the medical bag, wherein the sealed area of the medical bag comprises:
a first extension member arranged adjacent the first side of the medical bag;
a second extension member arranged adjacent the second side of the medical bag; and
a recess disposed between the first extension member and the second extension member; and
a suspension hole disposed in the sealed area of the medical bag between the fluid receiving area and the distal end of the medical bag.

12. The medical bag according to claim 11, wherein the at least one tube is bonded between the first resin sheet and the second resin sheet.

13. The medical bag according to claim 11, wherein the suspension hole is disposed in the sealed area in a center of the medical bag between the recess and the fluid receiving volume.

14. The medical bag according to claim 13, wherein the first extension member and the second extension member comprise a plurality of dimples formed in at least one of the first resin sheet and the second resin sheet.

15. The medical bag according to claim 14, wherein the dimples comprise a plurality of domed convex portions extending from the first resin sheet in a direction away from the second resin sheet.

16. The medical bag according to claim 14, wherein the medical bag is formed comprising a fold extending along the width of the medical bag such that a distal end of the the first extension member and a distal end overlap a portion of the fluid receiving volume.

17. The medical bag according to claim 16, wherein the fold is arranged distal to the suspension hole in the sealed area.

18. The medical bag according to claim 11, further comprising:
a connection port disposed between the first resin sheet and the second resin sheet adjacent the proximal end of the medical bag, the connection port fluidly connecting the fluid receiving volume with the at least one tube.

19. The medical bag according to claim 11, wherein a length of the first extension member is greater than one half of an outer circumference of the at least one tube.

20. The medical bag according to claim 11, wherein the first resin sheet and the second resin sheet are made from a thermoplastic material, and wherein the first resin sheet and the second resin sheet are fusion bonded together along the bonded edge.
